(19) United States
(12) Patent Application Publication
(10) Pub. No.: US 2018/0206774 A1
(43) Pub. Date: Jul. 26, 2018

(54) WEARABLE DEVICES FOR ASSISTING PARKINSON'S DISEASE PATIENTS

(71) Applicant: Intel Corporation, Santa Clara, CA (US)

(72) Inventor: Jinshi Huang, Fremont, CA (US)

(21) Appl. No.: 15/416,548

(22) Filed: Jan. 26, 2017

(51) Int. Cl. A61B 5/00; A61B 5/0205; A61B 5/11

(52) U.S. Cl. CPC A61B 5/4082; A61B 5/0022; A61B 5/0205; A61B 5/112; A61B 5/02416; A61B 5/6829; A61B 5/7264; A61B 5/7278; A61B 5/7282; A61B 5/6824

(57) ABSTRACT

A Parkinson's disease (PD) sensor system, including multiple inertial sensors and a heart rate monitor, may be used to detect PD symptoms, including Freezing of Gait (FoG). The PD sensor system may include a wrist-mounted accelerometer and heart rate monitor, and additional inertial sensors at other parts of the patient's body. The FoG detection classifier is implemented as an on-device neural network that will analyze data collected from the inertial sensors and the patient's heart rate to detect FoG events, and simultaneously uses the data to update the FoG event detection specific to the patient's PD symptoms. This self-learning neural network enables a personalized and optimized solution specific to each patient's PD symptoms and disease progression. Once a FoG event is detected, the sensor system may notify a concerned party or may activate an emergency response request.

### TECHNICAL FIELD

Embodiments described herein generally relate to wearable sensors.

### BACKGROUND

Parkinson's disease (PD) is a progressive degenerative disorder of the brain that inhibits the coordination of movement. Symptoms include tremor, stiffness, slowness of movement and instability, which affect the patient's ability to perform daily activities. About 10 million people worldwide are living with PD. Some treatments of PD include physical and occupational therapy. Various devices and technology have also been developed to assist the daily lives of the PD patients, including technology to assist in eating, sitting, dressing, bathing, toileting, and other activities. However, because PD symptoms are unique to each patient, it is difficult to create a technological solution that will work for every patient. Similarly, because PD symptoms progressively increase in severity, it is difficult to create a technological solution that will work for a single PD patient at multiple stages of the PD symptoms. Many existing devices or technology may only help a small subset of all PD patients, and may only help at specific stages of the disease. It is desirable to provide improved technology to assist many different PD patients throughout multiple progressive stages of PD symptoms.

### BRIEF DESCRIPTION OF THE DRAWINGS

FIG. 1 is a perspective diagram of a PD patient, in accordance with at least one embodiment.

FIG. 2 is a block diagram of a PD sensor network, in accordance with at least one embodiment.

FIG. 3 is a block diagram of a PD sensor communication system, in accordance with at least one embodiment.

FIG. 4 is a graph of acceleration and heart rate for an instructed stop, in accordance with at least one embodiment.

FIG. 5 is a graph of acceleration and heart rate for a FoG event, in accordance with at least one embodiment.

FIG. 6 is a flow chart for acceleration data feature extraction, in accordance with at least one embodiment.

FIG. 7 is a flow chart for heart rate data feature extraction, in accordance with at least one embodiment.

FIG. 8 is a block diagram for a wrist-worn FoG detection device, in accordance with at least one embodiment.

FIG. 9 is a block diagram for neural network training, in accordance with at least one embodiment.

FIG. 10 is a block diagram for neural network self-learning, in accordance with at least one embodiment.

FIG. 11 is a neural network self-learning graph, in accordance with at least one embodiment.

FIG. 12 is a block diagram of PD safety response services, in accordance with at least one embodiment.

FIG. 13 is a block diagram illustrating a FoG event detection system in the example form of an electronic device, according to an example embodiment.

### DESCRIPTION OF EMBODIMENTS

A Parkinson's disease (PD) sensor system, including multiple inertial sensors and a heart rate monitor, provides various technical solutions to the technical problems facing PD patient care. A common problem facing PD patients is known as Freezing of Gait (FoG), which manifests itself as sudden incapability of walking or movement. If not detected and addressed immediately, the patient may be in danger of falling, where a fall could cause major injury or death.

A PD sensor system may be used to detect FoG events. The PD sensor system may include a wrist-mounted accelerometer and heart rate monitor, and several inertial sensors at other parts of the patient's body. The FoG detection classifier is implemented as an on-device neural network that analyzes data collected from the inertial sensors and the patient's heart rate to detect FoG events, and simultaneously uses the data to update the FoG event detection specific to the patient's PD symptoms. The neural network is trained initially by a cloud-based PD symptom knowledge builder. After that, the neural network will adapt to the new conditions by self-learning using data collected from the patient, and will continue this self-learning as the PD progresses to each new stage. This self-learning neural network enables a personalized and optimized solution specific to each patient's PD symptoms and disease progression.

The PD sensor system may include a communication subsystem. Once a FoG event is detected, the communication subsystem is launched to notify the concerned parties (e.g., doctor, family). Additional services may be invoked by the communication subsystem, such as connecting to nearby devices to generate an alarm or to activate an emergency response request (e.g., ambulance request).

The following description and the drawings sufficiently illustrate specific embodiments to enable those skilled in the art to understand the specific embodiment. Other embodiments may incorporate structural, logical, electrical, process, and other changes. Portions and features of various embodiments may be included in, or substituted for, those of other embodiments. Embodiments set forth in the claims encompass all available equivalents of those claims.

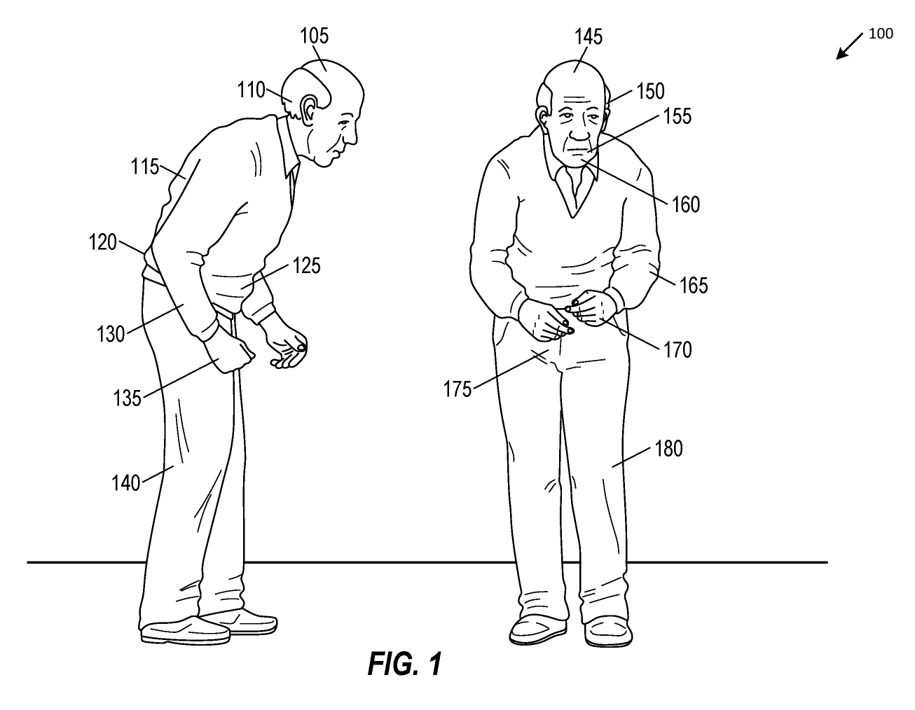

FIG. 1 is a perspective diagram of a PD patient 100, in accordance with at least one embodiment. Each PD patient 100 may exhibit one or more symptoms of PD. PD symptoms may include cognitive changes 105, such as mental slowing or progressive dementia. PD may result in a mood disorder 110, such as apathy, depression, or anxiety. Bodily symptoms of PD may include excessive sweating 115 (e.g., diaphoresis), fixed posture 120, constipation 125, reduced arm swing 130, or micrographia 135. In addition to FoG events, PD may result in a gait disturbance 140, which may include start hesitation, short shuffling steps, or other variations in gait.

PD may result in sleep disturbance 145, which may include insomnia, nightmares, or sleep walking. Facial symptoms of PD may include reduced facial expression 150 (e.g., reduced blinking or “Parkinsonian stare”), drooling 155 (e.g., sialorrhoea), or quiet and monotonous speech 160. PD may result in stiff arms 165, such as “lead-pipe rigidity” or positive or negative cogwheeling, and may result in a hand rest tremor 170. PD may result in urinary disorders 175, such as urinary retention, urinary infrequency, or impotence. PD may also result in postural instability or falls 180, such as FoG events.

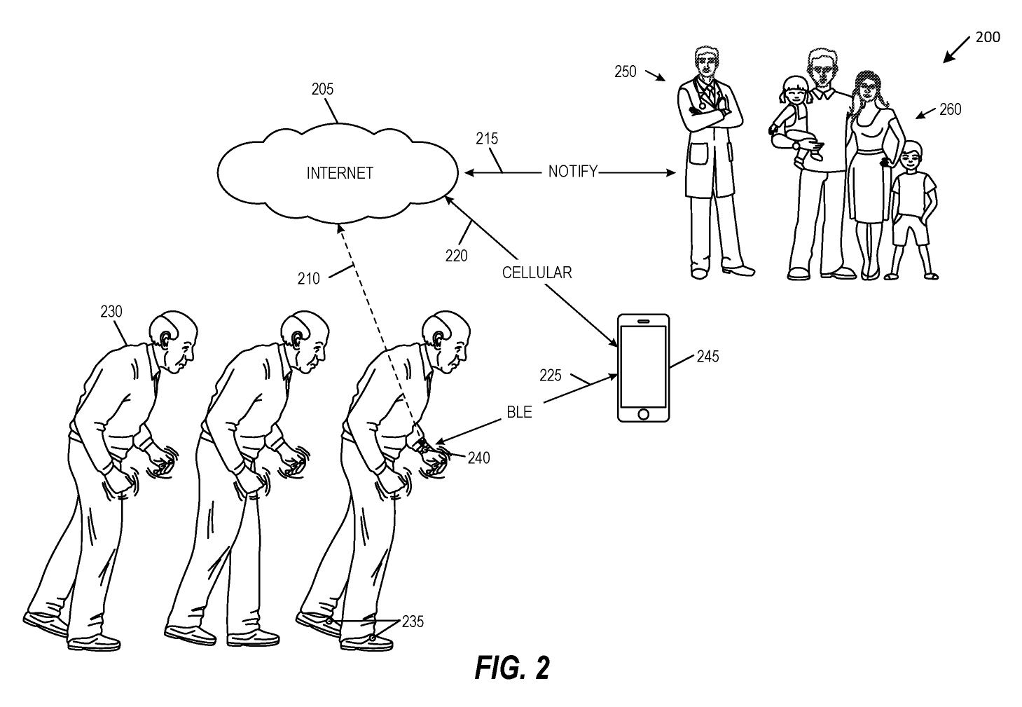

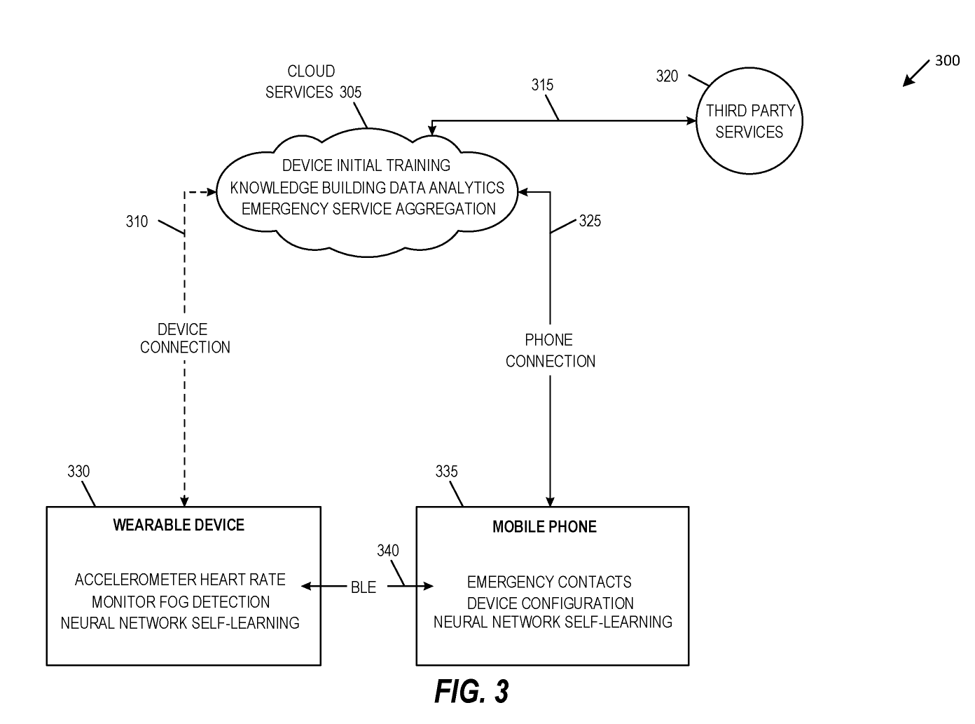

FIG. 2 is a block diagram of a PD sensor network 200, in accordance with at least one embodiment. A PD patient 230 may use a PD sensor network 200 to detect FoG events, improve the FoG event detection, improve the FoG event detection neural network, and notify a doctor 250 or family 260. The PD patient 230 wears at least a portion of the PD sensor network 200, including one or more inertial ankle sensors 235 and a wrist-worn device 240, where the wrist-worn device 240 includes accelerometer and heart rate monitoring. Data from the inertial ankle sensors 235 and wrist-worn device 240 is aggregated and communicated to another device. In an embodiment, aggregated data is communicated through a wireless channel 210 to the internet 205, where a notification may be communicated through a notification channel 215 to a doctor 250 or to family members 260. In an embodiment, aggregated data is communicated through a local wireless channel 225 (e.g., Bluetooth Low Energy (BLE)) to a mobile electronic device 245 such as a smartphone, which may be communicated through a cellular channel 220 to the internet 205 and onto a doctor 250 or family members 260. The FoG event detection neural network maybe implemented as on-device neural network on the wrist-worn device 240 or on the mobile electronic device 245. Additional details of the wrist-worn device 240 and mobile electronic device 245 are shown in FIG. 3 below.

FIG. 3 is a block diagram of a PD sensor communication system 300, in accordance with at least one embodiment. The sensor communication system 300 may be used for FoG detection and management. A wearable device 330 may collect and analyze the sensor data and heart rate variation to identify a FoG event and to update the FoG event detection neural network. When the wearable device 330 identifies a FoG event, it may send an alert through a communication channel 340 to a companion mobile phone 335. The mobile phone 335 may provide a PD patient or PD care provider with the ability to add or modify emergency contacts, to modify the configuration of the mobile phone 335 or of the wearable device 330, or to update the FoG event detection neural network. The wearable device 330 may provide raw data or FoG event alerts through an optional wearable device communication channel 310 (e.g., Wi-Fi channel, cellular communication channel) to cloud services 305. Similarly, the mobile phone 335 may provide raw data or FoG event alerts through a mobile phone communication channel 325 (e.g., Wi-Fi channel, cellular communication channel) to cloud services 305. Cloud services 305 may be used to manage initial neural network training, further data analysis in the form of knowledge-building data analytics, or emergency services aggregation. The cloud services 305 may be connected through a cloud communication channel 315 to one or more third-party services 320, such as services used to notify concerned parties or to activate an emergency response request.

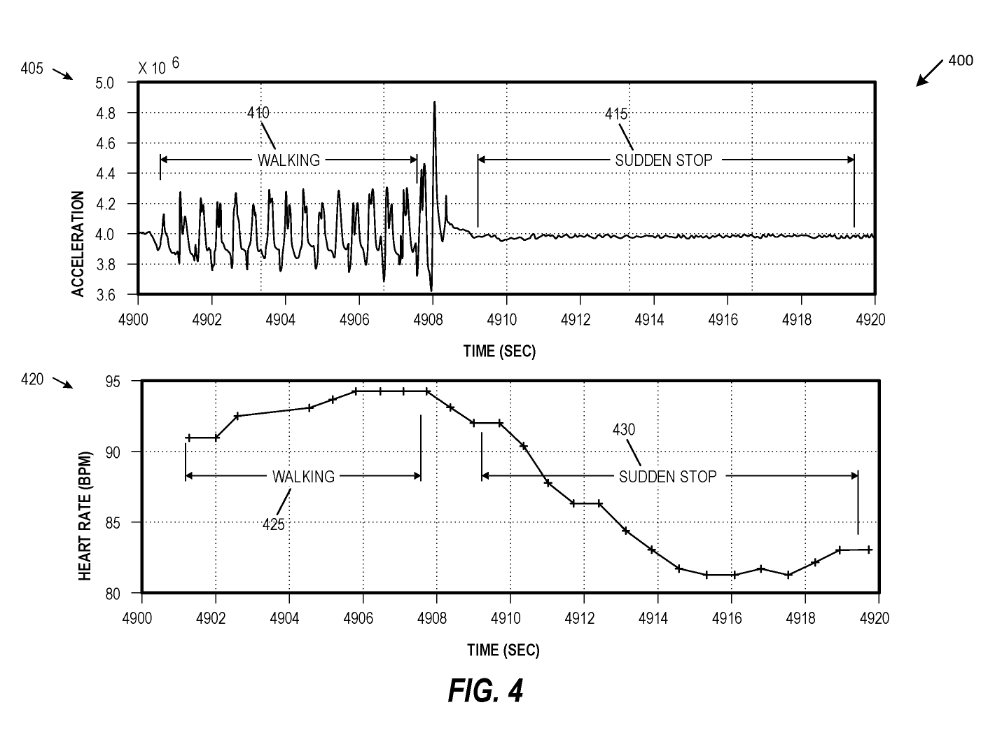

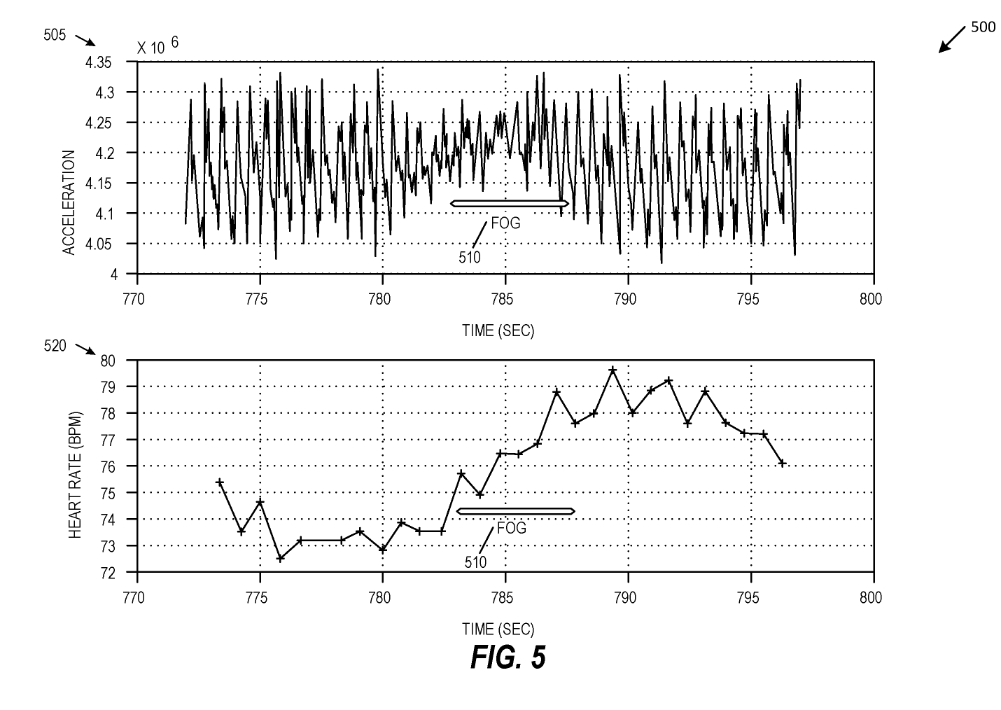

FIG. 4 is a graph of acceleration and heart rate for an instructed stop 400, in accordance with at least one embodiment. The graphs for the instructed stop 400 include an acceleration data graph 405 and a heart rate graph 420. The graphs for the instructed stop 400 show accelerometer data and heart rate samples for an instructed walking stop (i.e., a stop not caused by a FoG event). Acceleration graph 405 exhibits a rhythmic pattern during a normal walking. Acceleration graph 405 includes a walking portion 410 and a sudden stop portion 415. An instructed stop that is not caused by a FoG event includes a decrease in heart rate. Accordingly, the heart rate graph 420 depicts an increased heart rate during a walking portion 425 and a decreased heart rate during a sudden stop portion 430. The graphs for the instructed stop 400 are shown to contrast with data for a FoG event shown in FIG. 5, below.

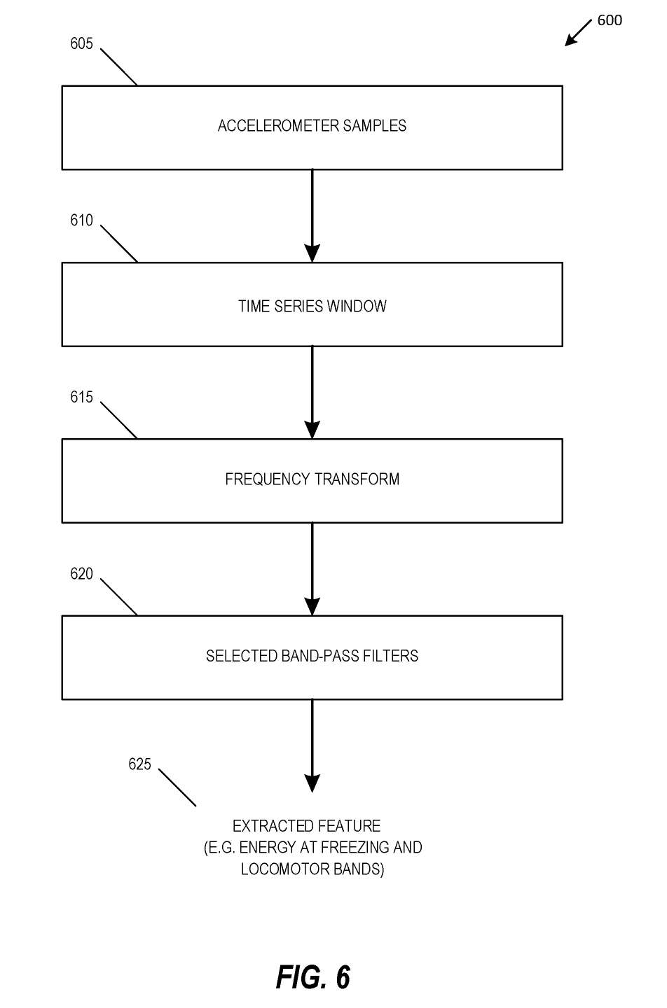

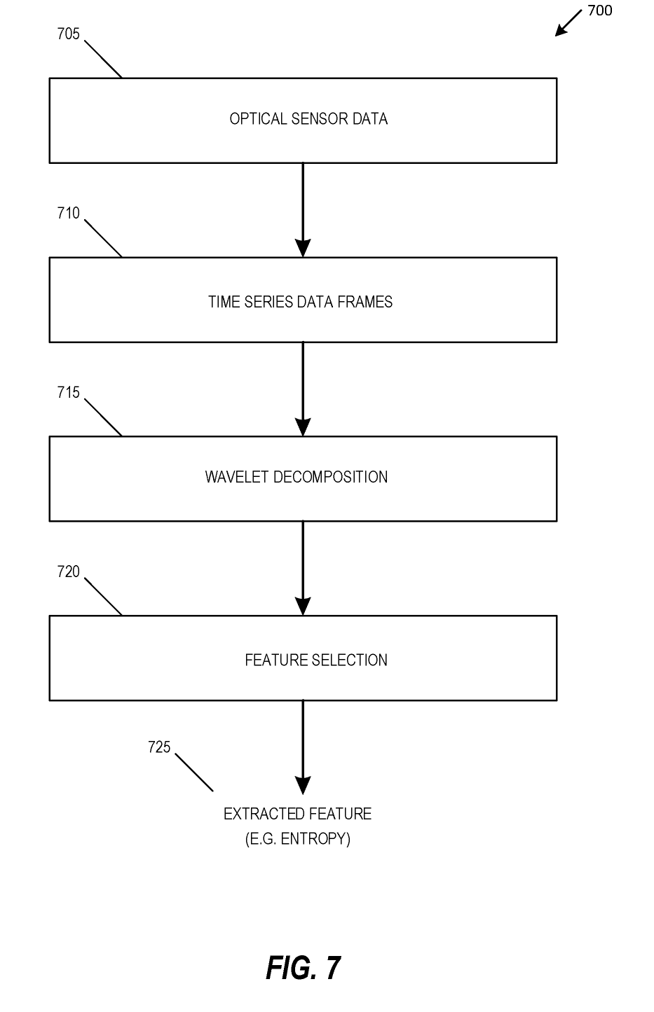

FIG. 5 is a graph of acceleration and heart rate for a FoG event 500, in accordance with at least one embodiment. The graphs for the FoG event 500 include an acceleration data graph 505 and a heart rate graph 520. Acceleration graph 505 shows accelerometer data samples before, during, and after a FoG event 510. Acceleration graph 505 exhibits a rhythmic pattern before the FoG event 510. Immediately prior to the FoG event 510, the acceleration graph 505 shows a significant increase of energy in the frequency band between 3 Hz and 8 Hz. Similarly, heart rate graph 520 shows heart rate samples before, during, and after the FoG event 510. Immediately prior to the FoG event 510, the heart rate starts to increase, and continues to increase during and shortly after the FoG event 510. This is in contrast with the decreased heart rate during an instructed stop portion 430 shown in FIG. 4. By detecting the combination of changes in acceleration and heart rate, a FoG event detection system may identify a FoG event and distinguish it from a voluntary or instructed stop. FoG event detection system may analyze accelerometer and heart rate to extract FoG event features, such as shown in FIG. 6 and FIG. 7.

FIG. 6 is a flow chart for acceleration data feature extraction 600, in accordance with at least one embodiment. Acceleration data feature extraction 600 may be used to process accelerometer samples 605 to extract features before the data is fed to a FoG neural network. Accelerometer samples 605 may be framed by a time series window 610. The windowed data is then transformed into the frequency domain 615, such as by using a fast Fourier transform or other frequency domain conversion algorithm. The frequency data is then filtered by selected bandpass filters 620, such as using a bandpass filter with a passband at known FoG event freezing and locomotor frequencies of 3 Hz to 8 Hz. The output of the bandpass filtering 620 includes an extracted accelerometer feature 625, where the extracted accelerometer feature 625 indicates that the accelerometer data is consistent with a FoG event.

FIG. 7 is a flow chart for heart rate data feature extraction 700, in accordance with at least one embodiment. Heart rate data feature extraction 700 may be used to process heart rate samples, such as heart rate samples received from optical sensor data 705. Optical sensor data 705 may be framed by time series data frames 710. A wavelet decomposition 715 is applied to the framed data, and a feature selection 720 is applied to identify extracted heart rate features 725. The extracted heart rate features 725 indicate that the heart rate data is consistent with a FoG event, such as indicating a significant increase in heart rate or an unpredictable heart rate variability (e.g., heart rate entropy).

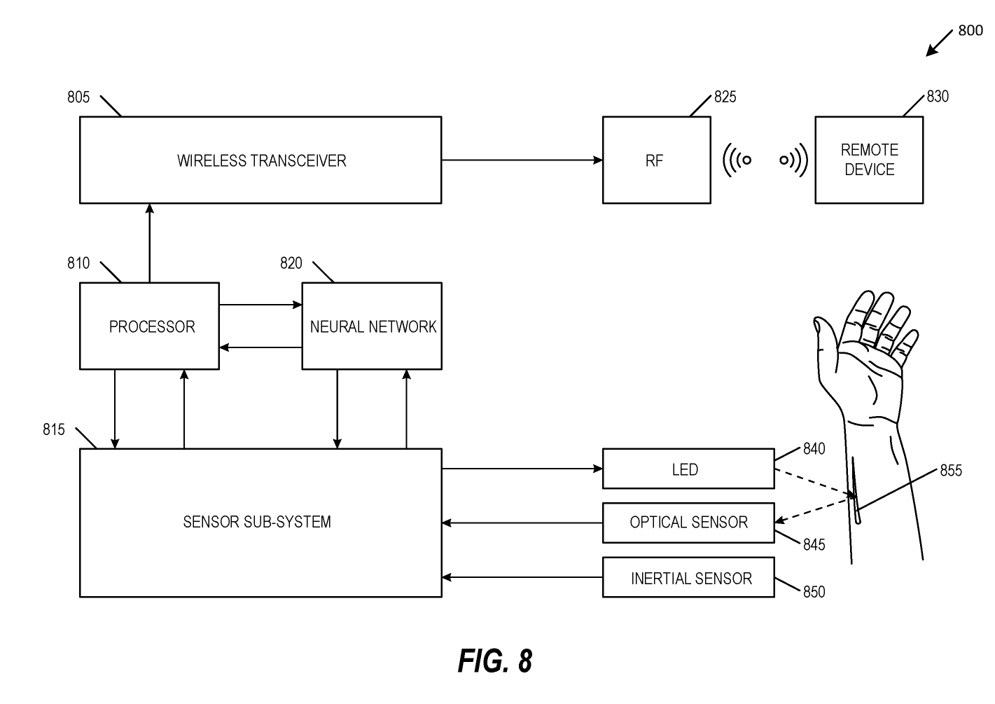

FIG. 8 is a block diagram for a wrist-worn FoG detection device 800, in accordance with at least one embodiment. Device 800 may include an LED 840, an optical sensor 845, and an inertial sensor 850. The LED 840 emits a light onto the user's skin 855. Reflected light is detected by the optical sensor 845 on the device, which is then digitized and fed through a sensor sub-system 815 to a processor 810 and neural network 820. Similarly, inertial data from the inertial sensor 850 is digitized and fed through sensor subsystem 815 to the processor 810 and neural network 820. Device 800 may include additional ankle inertial sensors (not shown) to provide additional inertial data. Both inertial sensor data and the average heart rate over time are analyzed by the neural network 820 to identify FoG events. When a FoG event is detected, a signal is sent through a wireless transceiver 805 to a radio frequency front-end 825 to a remote device 830, such as an alert sent to a doctor or family member.

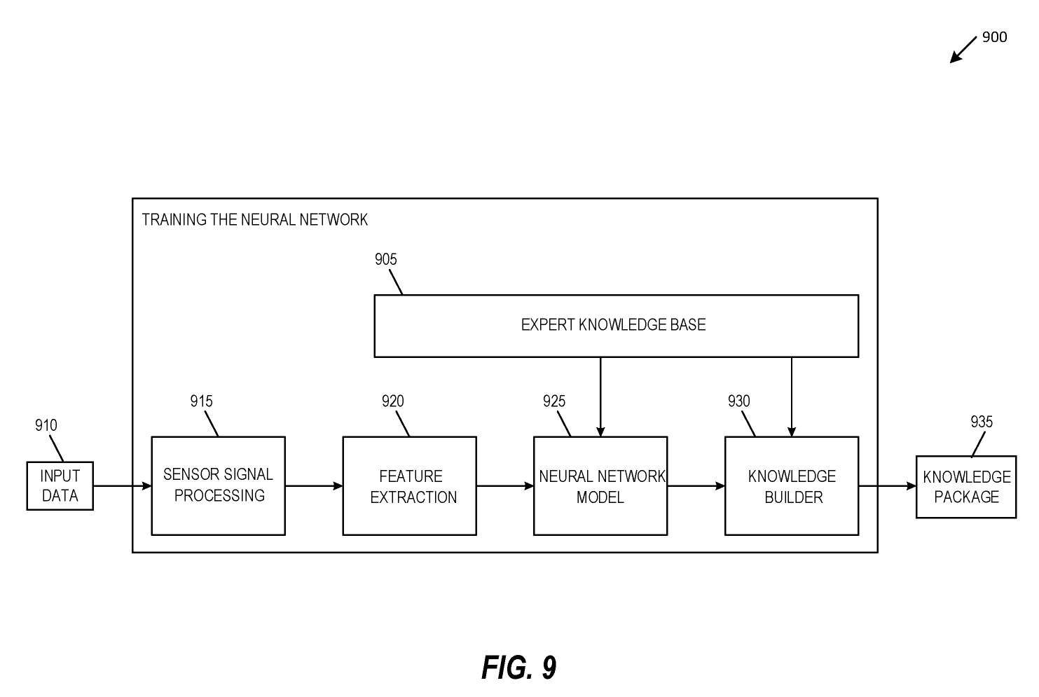

FIG. 9 is a block diagram for neural network training 900, in accordance with at least one embodiment. The neural network training 900 inside the device will be initially trained by an expert knowledge base 905, where the expert knowledge base 905 includes historical sensor data and heart rate sample data associated with a FoG event. The knowledge base 905 is used in the neural network model 925 and knowledge builder 930, and results in an output knowledge package 935. The knowledge package 935 includes process flows and software for the processor and neural network within the FoG event detection device, which may be installed on the FoG event detection device prior to its initial use. The process flows and software on the processor and neural network are updated based on additional data received from the user. For example, new input data 910 is provided to sensor signal processing 915, additional FoG event features are extracted 920, and these FoG event features are used to update the neural network model 925. The updated FoG event data may be used by the knowledge builder 930 to generate an updated knowledge package 935, which may be used to revise process flows and software on the processor and neural network within the FoG event detection device.

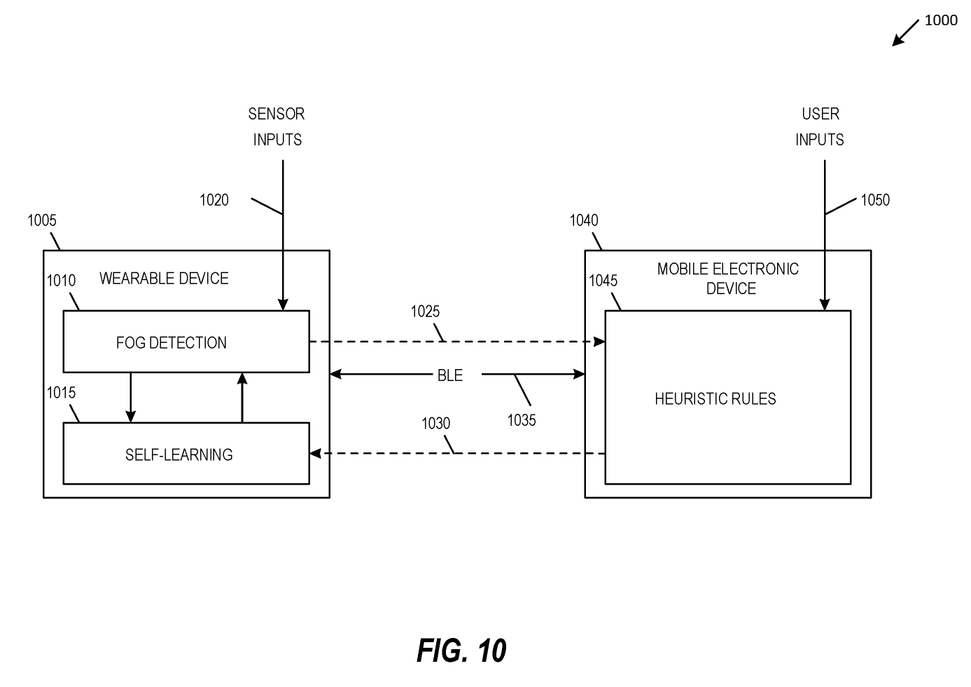

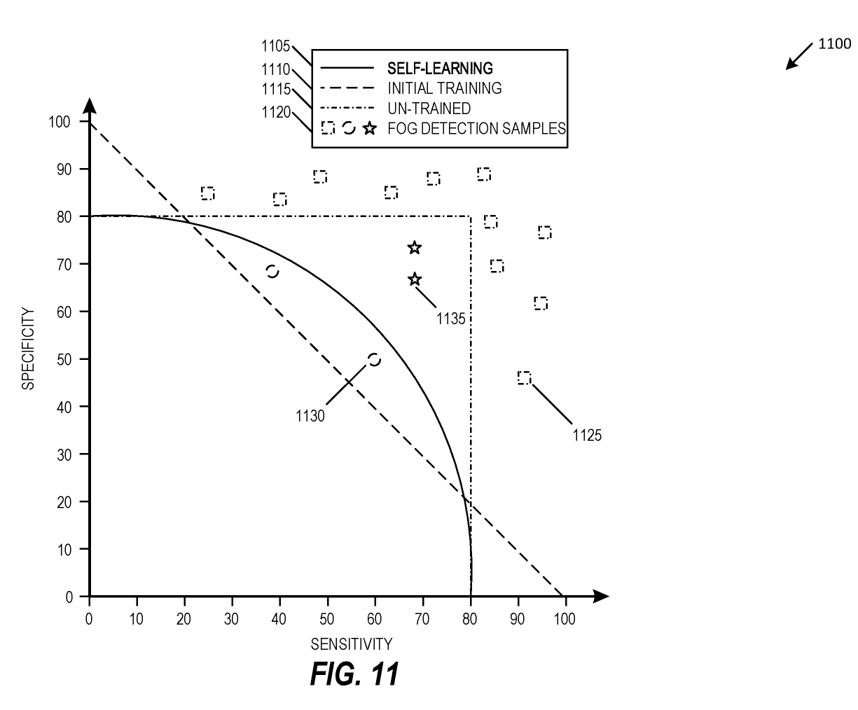

FIG. 10 is a block diagram for neural network self-learning 1000, in accordance with at least one embodiment. Because PD is a progressive disease, it will occasionally transition to new stages, and the FoG event detection device needs to be able to adapt to the new conditions in order to provide reliable and long-term FoG detection. This is achieved by using the self-learning capability 1015 on the wearable device 1005. The wearable device 1005 receives sensor inputs 1020 into FoG detection 1010, and FoG detection 1010 interacts with self-learning 1015 to adapt to new sensor input data. The wearable device 1005 communicates with a mobile electronic device 1040, such as communicating over a BLE channel 1035. The mobile electronic device 1040 may include a set of heuristic rules 1045 used to enhance the ability of the wearable device 1005 to provide reliable and long-term FoG detection. In particular, the mobile electronic device 1040 may receive user inputs 1050, and may determine a set of heuristic rules 1045 that simplify the calculations within (i.e. increase the performance of) FoG detection 1010 or self-learning 1015. The mobile electronic device 1040 may communicate the heuristic rules 1045 may communicate with the self-learning 1015 over self-learning communication channel 1030. An example of the modification of self-learning 1015 is shown in FIG. 11 below.

FIG. 11 is a neural network self-learning graph 1100, in accordance with at least one embodiment. Each time a FoG event is detected, the new data is fed into the self-learning module. The self-learning module seeks to provide a good balance between sensitivity (i.e., detecting a FoG event) and specificity (i.e., avoiding false alarms). Self-learning graph 1100 shows a balance between this sensitivity and specificity. Initially, untrained data 1115 represents an in initial value of 80% specificity for sensitivity values from 0% to 80%. Initial training 1110 represents the tradeoff between specificity and sensitivity, where the change in specificity is inversely proportionate with the change in sensitivity. As additional FoG event detection samples 1120 are provided, the self-learning neural network generates the self-learning curve 1105. Additional FoG event detection samples 1120 may be characterized based on the self-learning graph 1100. For example, samples 1125 may fall outside of the untrained data 1115 thresholds of 80% specificity and 80% sensitivity, and may be excluded as not qualifying as a FoG event. Similarly, samples 1130 may fall below the self-learning curve 1105, and may be excluded as insufficiently sensitive or specific to qualify as a FoG event. Some samples 1135 may fall above the self-learning curve 1105 yet below the untrained data 1115, and may be identified as FoG events. In addition to the use of new FoG data, the curves in the self-learning graph 1100 may be implemented as a semi-supervised learning neural network that is updated based on heuristic rules and user inputs from a mobile electronic device. The use of additional inputs from a mobile electronic device enables the FoG event detector to improve the accuracy of the sensitivity and specificity curves.

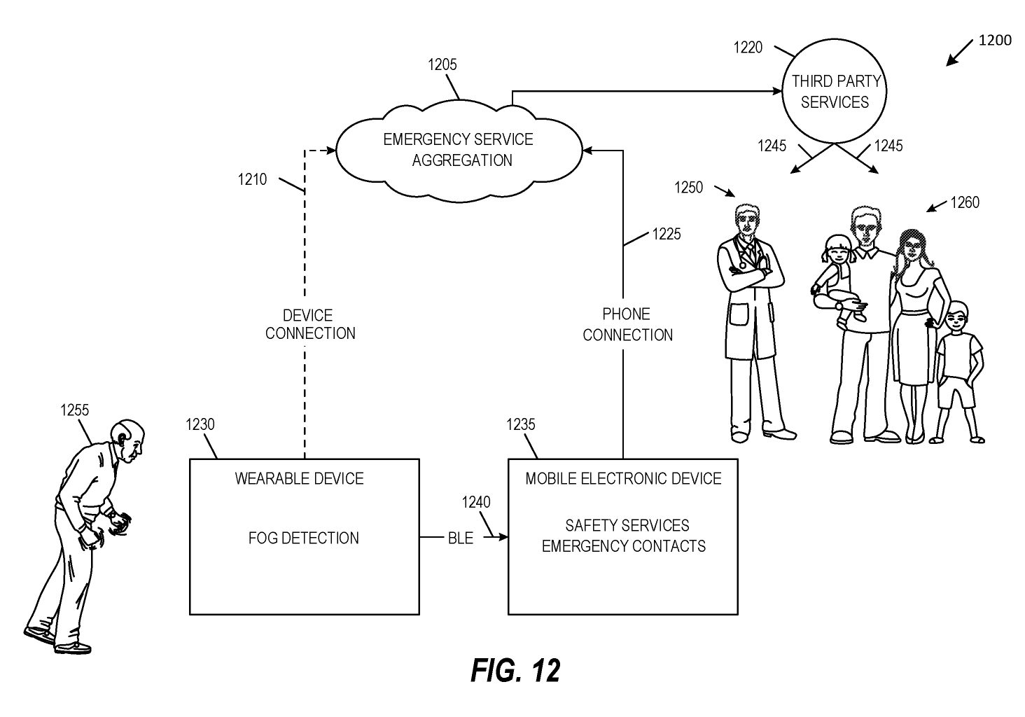

FIG. 12 is a block diagram of PD safety response services 1200, in accordance with at least one embodiment. When a PD patient 1255 experiences a FoG event, the wearable device 1230 may use a local wireless communication channel 1240 to alert the mobile electronic device 135. The mobile electronic device 1235 may communicate through a mobile device connection 1225 to an emergency service aggregation 1205, which may invoke one or more third party services 1220 to send a FoG event notification 1245 to a doctor 1250 or a family member 1260. In an embodiment, the wearable device 1230 may communicate through an optional wearable device connection 1210 to the emergency service aggregation 1205 and to third party services 1220 to send a FoG event notification 1245 to a doctor 1250 or a family member 1260.

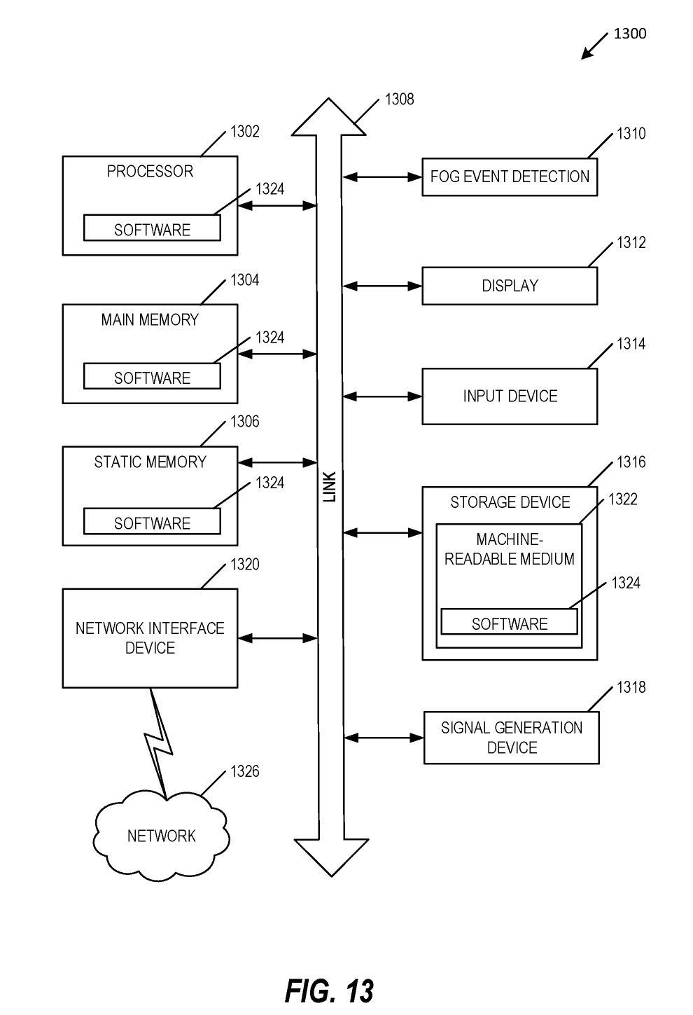

FIG. 13 is a block diagram illustrating a FoG event detection system in the example form of an electronic device 1300, within which a set or sequence of instructions may be executed to cause the machine to perform any one of the methodologies discussed herein, according to an example embodiment. Electronic device 1300 may also represent the devices shown in FIGS. 1-2. In alternative embodiments, the electronic device 1300 operates as a standalone device or may be connected (e.g., networked) to other machines. In a networked deployment, the electronic device 1300 may operate in the capacity of either a server or a client machine in server-client network environments, or it may act as a peer machine in peer-to-peer (or distributed) network environments. The electronic device 1300 may be an integrated circuit (IC), a portable electronic device, a personal computer (PC), a tablet PC, a hybrid tablet, a personal digital assistant (PDA), a mobile telephone, or any electronic device 1300 capable of executing instructions (sequential or otherwise) that specify actions to be taken by that machine to detect a user input. Further, while only a single electronic device 1300 is illustrated, the terms “machine” or “electronic device” shall also be taken to include any collection of machines or devices that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to execute instructions, individually or jointly, to perform any one or more of the methodologies discussed herein.

Example electronic device 1300 includes at least one processor 1302 (e.g., a central processing unit (CPU), a graphics processing unit (GPU) or both, processor cores, compute nodes, etc.), a main memory 1304 and a static memory 1306, which communicate with each other via a link 1308 (e.g., bus).

The electronic device 1300 includes a FoG event detection system 1310, where the FoG event detection system 1310 may include a heart rate sensor and one or more inertial sensors as described above. The electronic device 1300 may further include a display unit 1312, where the display unit 1312 may include a single component that provides a user-readable display and a protective layer, or another display type. The electronic device 1300 may further include an input device 1314, such as a pushbutton, a keyboard, an NFC card reader, or a user interface (UI) navigation device (e.g., a mouse or touch-sensitive input). The electronic device 1300 may additionally include a storage device 1316, such as a drive unit. The electronic device 1300 may additionally include a signal generation device 1318 to provide audible or visual feedback, such as a speaker to provide an audible feedback or one or more LEDs to provide a visual feedback. The electronic device 1300 may additionally include a network interface device 1320, and one or more additional sensors (not shown), such as a global positioning system (GPS) sensor, compass, accelerometer, or other sensor.

The storage device 1316 includes a machine-readable medium 1322 on which is stored one or more sets of data structures and instructions 1324 (e.g., software) embodying or utilized by any one or more of the methodologies or functions described herein. The instructions 1324 may also reside, completely or at least partially, within the main memory 1304, static memory 1306, and/or within the processor 1302 during execution thereof by the electronic device 1300. The main memory 1304, static memory 1306, and the processor 1302 may also constitute machine-readable media.

While the machine-readable medium 1322 is illustrated in an example embodiment to be a single medium, the term “machine-readable medium” may include a single medium or multiple media (e.g., a centralized or distributed database, and/or associated caches and servers) that store the one or more instructions 1324. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1324 may further be transmitted or received over a communications network 1326 using a transmission medium via the network interface device 1320 utilizing any one of a number of well-known transfer protocols (e.g., HTTP). Examples of communication networks include a local area network (LAN), a wide area network (WAN), the Internet, mobile telephone networks, and wireless data networks (e.g., Wi-Fi, NFC, Bluetooth, Bluetooth LE, 3G, 3G LTE/LTE-A, WiMAX networks, etc.). The term “transmission medium” shall be taken to include any intangible medium that is capable of storing, encoding, or carrying instructions for execution by the machine, and includes digital or analog communications signals or other intangible medium to facilitate communication of such software.

To better illustrate the method and apparatuses disclosed herein, a non-limiting list of embodiments is provided here.

Example 1 is a Freezing of Gait (FoG) event detection apparatus comprising: a heart rate detection device to generate a heart rate sample; a first inertial sensor to generate a first plurality of inertial measurements; and a processor to identify a FoG event based on the heart rate sample and on the first plurality of inertial measurements.

In Example 2, the subject matter of Example 1 optionally includes the processor further to: extract a FoG inertial feature; and identify the FoG event based on the FoG inertial feature.

In Example 3, the subject matter of Example 2 optionally includes the processor further to: extract a FoG heart rate feature; and identify the FoG event based on the FoG heart rate feature.

In Example 4, the subject matter of Example 3 optionally includes the processor further to: identify a FoG inertial event time associated with the FoG inertial feature; identify a FoG heart rate event time associated with the FoG heart rate feature; and identify the FoG event based on a time overlap between the FoG inertial event time and the FoG heart rate event time.

In Example 5, the subject matter of any one or more of Examples 3-4 optionally include the processor further to analyze the extracted FoG inertial feature and extracted FoG heart rate feature in a neural network model to identify the FoG event.

In Example 6, the subject matter of Example 5 optionally includes wherein the neural network model includes a FoG inertial threshold and a FoG heart rate threshold.

In Example 7, the subject matter of Example 6 optionally includes wherein the FoG event is identified when the FoG inertial feature exceeds the FoG inertial threshold and when the FoG heart rate feature exceeds the FoG heart rate threshold.

In Example 8, the subject matter of any one or more of Examples 6-7 optionally include wherein the neural network FoG inertial threshold and the FoG heart rate threshold are based on an expert knowledge base initial model.

In Example 9, the subject matter of any one or more of Examples 6-8 optionally include the processor further to update the neural network FoG inertial threshold based on the FoG inertial feature.

In Example 10, the subject matter of any one or more of Examples 6-9 optionally include the processor further to update the neural network FoG heart rate threshold based on the FoG heart rate feature.

In Example 11, the subject matter of any one or more of Examples 6-10 optionally include the processor further to: receive a heuristic inertial rule from a mobile electronic device; and update the neural network FoG inertial threshold based on the received heuristic inertial rule.

In Example 12, the subject matter of any one or more of Examples 6-11 optionally include the processor further to: receive a heuristic heart rate rule from a mobile electronic device; and update the neural network FoG heart rate threshold based on the received heuristic heart rate rule.

In Example 13, the subject matter of any one or more of Examples 1-12 optionally include the processor further to: transform the first plurality of inertial measurements from the time domain into a plurality of inertial measurement amplitudes in the frequency domain; and identify the FoG event based on a frequency component within the plurality of inertial measurement amplitudes.

In Example 14, the subject matter of Example 13 optionally includes the processor further to sample the first plurality of inertial measurements based on a time series window prior to transforming the first plurality of inertial measurements.

In Example 15, the subject matter of Example 14 optionally includes the processor further to apply a frequency filter to the plurality of inertial measurement amplitudes to extract a FoG inertial feature.

In Example 16, the subject matter of Example 15 optionally includes wherein the frequency filter includes a bandpass filter.

In Example 17, the subject matter of Example 16 optionally includes wherein the bandpass filter includes a passband from 3 Hz to 8 Hz.

In Example 18, the subject matter of any one or more of Examples 1-17 optionally include wherein the heart rate detection device includes an optical heart rate sensor to generate optical heart rate sensor data.

In Example 19, the subject matter of Example 18 optionally includes the processor further to: decompose the optical heart rate sensor data into a heart rate wavelet; and extract a FoG heart rate feature based on the heart rate wavelet.

In Example 20, the subject matter of any one or more of Examples 1-19 optionally include the processor further to determine a heart rate increase based on the heart rate sample and based on a plurality of heart rate historical data.

In Example 21, the subject matter of any one or more of Examples 1-20 optionally include wherein the heart rate detection device and the first inertial sensor are included within a device worn on a user wrist.

In Example 22, the subject matter of Example 21 optionally includes a second inertial sensor to generate a second plurality of inertial measurements, wherein the processor identifying the FoG event is further based on the second plurality of inertial measurements.

In Example 23, the subject matter of Example 22 optionally includes wherein the second inertial sensor is included within a device worn on a user ankle.

In Example 24, the subject matter of any one or more of Examples 1-23 optionally include the processor further to notify a patient care provider in response to identifying the FoG event.

Example 25 is a Freezing of Gait (FoG) event detection method comprising: receiving a heart rate sample from a heart rate detection device; receiving a first plurality of inertial measurements from a first inertial sensor; and identifying a FoG event based on the heart rate sample and on the first plurality of inertial measurements.

In Example 26, the subject matter of Example 25 optionally includes wherein identifying the FoG event includes: extracting a FoG inertial feature; and identifying the FoG event based on the FoG inertial feature.

In Example 27, the subject matter of Example 26 optionally includes wherein identifying the FoG event includes: extracting a FoG heart rate feature; and identifying the FoG event based on the FoG heart rate feature.

In Example 28, the subject matter of Example 27 optionally includes wherein identifying the FoG event includes: identifying a FoG inertial event time associated with the FoG inertial feature; identifying a FoG heart rate event time associated with the FoG heart rate feature; and identifying the FoG event based on a time overlap between the FoG inertial event time and the FoG heart rate event time.

In Example 29, the subject matter of any one or more of Examples 27-28 optionally include wherein identifying the FoG event includes analyzing the extracted FoG inertial feature and extracted FoG heart rate feature in a neural network model to identify the FoG event.

In Example 30, the subject matter of Example 29 optionally includes wherein the neural network model includes a FoG inertial threshold and a FoG heart rate threshold.

In Example 31, the subject matter of Example 30 optionally includes wherein the FoG event is identified when the FoG inertial feature exceeds the FoG inertial threshold and when the FoG heart rate feature exceeds the FoG heart rate threshold.

In Example 32, the subject matter of any one or more of Examples 30-31 optionally include wherein the neural network FoG inertial threshold and the FoG heart rate threshold are based on an expert knowledge base initial model.

In Example 33, the subject matter of any one or more of Examples 30-32 optionally include updating the neural network FoG inertial threshold based on the FoG inertial feature.

In Example 34, the subject matter of any one or more of Examples 30-33 optionally include updating the neural network FoG heart rate threshold based on the FoG heart rate feature.

In Example 35, the subject matter of any one or more of Examples 30-34 optionally include receiving a heuristic inertial rule from a mobile electronic device; and updating the neural network FoG inertial threshold based on the received heuristic inertial rule.

In Example 36, the subject matter of any one or more of Examples 30-35 optionally include receiving a heuristic heart rate rule from a mobile electronic device; and updating the neural network FoG heart rate threshold based on the received heuristic heart rate rule.

In Example 37, the subject matter of any one or more of Examples 26-36 optionally include transforming the first plurality of inertial measurements from the time domain into a plurality of inertial measurement amplitudes in the frequency domain; and identifying the FoG event based on a frequency component within the plurality of inertial measurement amplitudes.

In Example 38, the subject matter of Example 37 optionally includes sampling the first plurality of inertial measurements based on a time series window prior to transforming the first plurality of inertial measurements.

In Example 39, the subject matter of Example 38 optionally includes applying a frequency filter to the plurality of inertial measurement amplitudes to extract a FoG inertial feature.

In Example 40, the subject matter of Example 39 optionally includes wherein the frequency filter includes a bandpass filter.

In Example 41, the subject matter of Example 40 optionally includes wherein the bandpass filter includes a passband from 3 Hz to 8 Hz.

In Example 42, the subject matter of any one or more of Examples 27-41 optionally include wherein extracting the FoG heart rate feature includes determining a heart rate increase based on the heart rate sample and based on a plurality of heart rate historical data.

In Example 43, the subject matter of any one or more of Examples 27-42 optionally include wherein receiving the heart rate sample includes receiving optical heart rate sensor data from the heart rate detection device.

In Example 44, the subject matter of Example 43 optionally includes decomposing the optical heart rate sensor data into a heart rate wavelet, wherein extracting the FoG heart rate feature is based on the heart rate wavelet.

In Example 45, the subject matter of any one or more of Examples 25-44 optionally include wherein the heart rate detection device and the first inertial sensor are included within a device worn on a user wrist.

In Example 46, the subject matter of Example 45 optionally includes receiving a second plurality of inertial measurements from a second inertial sensor, wherein identifying the FoG event is further based on the second plurality of inertial measurements.

In Example 47, the subject matter of Example 46 optionally includes wherein the second inertial sensor is included within a device worn on a user ankle.

In Example 48, the subject matter of any one or more of Examples 25-47 optionally include notifying a patient care provider in response to identifying the FoG event.

Example 49 is at least one machine-readable medium including instructions, which when executed by a computing system, cause the computing system to perform any of the methods of Examples 25-48.

Example 50 is an apparatus comprising means for performing any of the methods of Examples 25-48.

Example 51 is at least one machine-readable storage medium, comprising a plurality of instructions that, responsive to being executed with processor circuitry of a computer-controlled device, cause the computer-controlled device to: receive a heart rate sample from a heart rate detection device; receive a first plurality of inertial measurements from a first inertial sensor; and identify a FoG event based on the heart rate sample and on the first plurality of inertial measurements.

In Example 52, the subject matter of Example 51 optionally includes the plurality of instructions further causing the computer-controlled device to: extract a FoG inertial feature; and identify the FoG event based on the FoG inertial feature.

In Example 53, the subject matter of Example 52 optionally includes the plurality of instructions further causing the computer-controlled device to: extract a FoG heart rate feature; and identify the FoG event based on the FoG heart rate feature.

In Example 54, the subject matter of Example 53 optionally includes the plurality of instructions further causing the computer-controlled device to: identify a FoG inertial event time associated with the FoG inertial feature; identify a FoG heart rate event time associated with the FoG heart rate feature; and identify the FoG event based on a time overlap between the FoG inertial event time and the FoG heart rate event time.

In Example 55, the subject matter of any one or more of Examples 53-54 optionally include the plurality of instructions further causing the computer-controlled device to analyze the extracted FoG inertial feature and extracted FoG heart rate feature in a neural network model to identify the FoG event.

In Example 56, the subject matter of Example 55 optionally includes wherein the neural network model includes a FoG inertial threshold and a FoG heart rate threshold.

In Example 57, the subject matter of Example 56 optionally includes wherein the FoG event is identified when the FoG inertial feature exceeds the FoG inertial threshold and when the FoG heart rate feature exceeds the FoG heart rate threshold.

In Example 58, the subject matter of any one or more of Examples 56-57 optionally include wherein the neural network FoG inertial threshold and the FoG heart rate threshold are based on an expert knowledge base initial model.

In Example 59, the subject matter of any one or more of Examples 56-58 optionally include the plurality of instructions further causing the computer-controlled device to update the neural network FoG inertial threshold based on the FoG inertial feature.

In Example 60, the subject matter of any one or more of Examples 56-59 optionally include the plurality of instructions further causing the computer-controlled device to update the neural network FoG heart rate threshold based on the FoG heart rate feature.

In Example 61, the subject matter of any one or more of Examples 56-60 optionally include the plurality of instructions further causing the computer-controlled device to: receive a heuristic inertial rule from a mobile electronic device; and update the neural network FoG inertial threshold based on the received heuristic inertial rule.

In Example 62, the subject matter of any one or more of Examples 56-61 optionally include the plurality of instructions further causing the computer-controlled device to: receive a heuristic heart rate rule from a mobile electronic device; and update the neural network FoG heart rate threshold based on the received heuristic heart rate rule.

In Example 63, the subject matter of any one or more of Examples 52-62 optionally include the plurality of instructions further causing the computer-controlled device to: transform the first plurality of inertial measurements from the time domain into a plurality of inertial measurement amplitudes in the frequency domain; and identify the FoG event based on a frequency component within the plurality of inertial measurement amplitudes.

In Example 64, the subject matter of Example 63 optionally includes the plurality of instructions further causing the computer-controlled device to sample the first plurality of inertial measurements based on a time series window prior to transforming the first plurality of inertial measurements.

In Example 65, the subject matter of Example 64 optionally includes the plurality of instructions further causing the computer-controlled device to apply a frequency filter to the plurality of inertial measurement amplitudes to extract a FoG inertial feature.

In Example 66, the subject matter of Example 65 optionally includes wherein the frequency filter includes a bandpass filter.

In Example 67, the subject matter of Example 66 optionally includes wherein the bandpass filter includes a passband from 3 Hz to 8 Hz.

In Example 68, the subject matter of any one or more of Examples 53-67 optionally include the plurality of instructions further causing the computer-controlled device to determine a heart rate increase based on the heart rate sample and based on a plurality of heart rate historical data.

In Example 69, the subject matter of any one or more of Examples 53-68 optionally include the plurality of instructions further causing the computer-controlled device to receive optical heart rate sensor data from the heart rate detection device.

In Example 70, the subject matter of Example 69 optionally includes the plurality of instructions further causing the computer-controlled device to decompose the optical heart rate sensor data into a heart rate wavelet, wherein extracting the FoG heart rate feature is based on the heart rate wavelet.

In Example 71, the subject matter of any one or more of Examples 51-70 optionally include wherein the heart rate detection device and the first inertial sensor are included within a device worn on a user wrist.

In Example 72, the subject matter of Example 71 optionally includes the plurality of instructions further causing the computer-controlled device to receive a second plurality of inertial measurements from a second inertial sensor, wherein identifying the FoG event is further based on the second plurality of inertial measurements.

In Example 73, the subject matter of Example 72 optionally includes wherein the second inertial sensor is included within a device worn on a user ankle.

In Example 74, the subject matter of any one or more of Examples 51-73 optionally include the plurality of instructions further causing the computer-controlled device to notify a patient care provider in response to identifying the FoG event.

Example 75 is a Freezing of Gait (FoG) event detection apparatus comprising: means for receiving a heart rate sample from a heart rate detection device; means for receiving a first plurality of inertial measurements from a first inertial sensor; and means for identifying a FoG event based on the heart rate sample and on the first plurality of inertial measurements.

In Example 76, the subject matter of Example 75 optionally includes wherein means for identifying the FoG event includes: means for extracting a FoG inertial feature; and means for identifying the FoG event based on the FoG inertial feature.

In Example 77, the subject matter of Example 76 optionally includes wherein means for identifying the FoG event includes: means for extracting a FoG heart rate feature; and means for identifying the FoG event based on the FoG heart rate feature.

In Example 78, the subject matter of Example 77 optionally includes wherein means for identifying the FoG event includes: means for identifying a FoG inertial event time associated with the FoG inertial feature; means for identifying a FoG heart rate event time associated with the FoG heart rate feature; and means for identifying the FoG event based on a time overlap between the FoG inertial event time and the FoG heart rate event time.

In Example 79, the subject matter of any one or more of Examples 77-78 optionally include wherein means for identifying the FoG event includes means for analyzing the extracted FoG inertial feature and extracted FoG heart rate feature in a neural network model to identify the FoG event.

In Example 80, the subject matter of Example 79 optionally includes wherein the neural network model includes a FoG inertial threshold and a FoG heart rate threshold.

In Example 81, the subject matter of Example 80 optionally includes wherein the FoG event is identified when the FoG inertial feature exceeds the FoG inertial threshold and when the FoG heart rate feature exceeds the FoG heart rate threshold.

In Example 82, the subject matter of any one or more of Examples 80-81 optionally include wherein the neural network FoG inertial threshold and the FoG heart rate threshold are based on an expert knowledge base initial model.

In Example 83, the subject matter of any one or more of Examples 80-82 optionally include means for updating the neural network FoG inertial threshold based on the FoG inertial feature.

In Example 84, the subject matter of any one or more of Examples 80-83 optionally include means for updating the neural network FoG heart rate threshold based on the FoG heart rate feature.

In Example 85, the subject matter of any one or more of Examples 80-84 optionally include means for receiving a heuristic inertial rule from a mobile electronic device; and means for updating the neural network FoG inertial threshold based on the received heuristic inertial rule.

In Example 86, the subject matter of any one or more of Examples 80-85 optionally include means for receiving a heuristic heart rate rule from a mobile electronic device; and means for updating the neural network FoG heart rate threshold based on the received heuristic heart rate rule.

In Example 87, the subject matter of any one or more of Examples 76-86 optionally include means for transforming the first plurality of inertial measurements from the time domain into a plurality of inertial measurement amplitudes in the frequency domain; and means for identifying the FoG event based on a frequency component within the plurality of inertial measurement amplitudes.

In Example 88, the subject matter of Example 87 optionally includes means for sampling the first plurality of inertial measurements based on a time series window prior to transforming the first plurality of inertial measurements.

In Example 89, the subject matter of Example 88 optionally includes means for applying a frequency filter to the plurality of inertial measurement amplitudes to extract a FoG inertial feature.

In Example 90, the subject matter of Example 89 optionally includes wherein the frequency filter includes a bandpass filter.

In Example 91, the subject matter of Example 90 optionally includes wherein the bandpass filter includes a passband from 3 Hz to 8 Hz.

In Example 92, the subject matter of any one or more of Examples 77-91 optionally include wherein means for extracting the FoG heart rate feature includes means for determining a heart rate increase based on the heart rate sample and based on a plurality of heart rate historical data.

In Example 93, the subject matter of any one or more of Examples 77-92 optionally include wherein means for receiving the heart rate sample includes means for receiving optical heart rate sensor data from the heart rate detection device.

In Example 94, the subject matter of Example 93 optionally includes means for decomposing the optical heart rate sensor data into a heart rate wavelet, wherein means for extracting the FoG heart rate feature is based on the heart rate wavelet.

In Example 95, the subject matter of any one or more of Examples 75-94 optionally include wherein the heart rate detection device and the first inertial sensor are included within a device worn on a user wrist.

In Example 96, the subject matter of Example 95 optionally includes means for receiving a second plurality of inertial measurements from a second inertial sensor, wherein means for identifying the FoG event is further based on the second plurality of inertial measurements.

In Example 97, the subject matter of Example 96 optionally includes wherein the second inertial sensor is included within a device worn on a user ankle.

In Example 98, the subject matter of any one or more of Examples 75-97 optionally include means for notifying a patient care provider in response to identifying the FoG event.

Example 99 is at least one machine-readable medium including instructions, which when executed by a machine, cause the machine to perform operations of any of the operations of Examples 1-98.

Example 100 is an apparatus comprising means for performing any of the operations of Examples 1-98.

Example 101 is a system to perform the operations of any of the Examples 1-98.

Example 102 is a method to perform the operations of any of the Examples 1-98.

The above detailed description includes references to the accompanying drawings, which form a part of the detailed description. The drawings show, by way of illustration, specific embodiments in which the embodiments described herein may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. In the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments may be combined with each other in various combinations or permutations. The scope should be determined with reference to the appended claims, along with the full scope of equivalents to which such claims are entitled.

1. A Freezing of Gait (FoG) event detection apparatus comprising:
a heart rate detection device to generate a heart rate sample;
a first inertial sensor to generate a first plurality of inertial measurements; and
a processor to identify a FoG event based on the heart rate sample and on the first plurality of inertial measurements.

2. The apparatus of claim 1, the processor further to:
extract a FoG inertial feature; and
identify the FoG event based on the FoG inertial feature.

3. The apparatus of claim 2, the processor further to:
extract a FoG heart rate feature; and
identify the FoG event based on the FoG heart rate feature.

4. The apparatus of claim 3, the processor further to analyze the extracted FoG inertial feature and extracted FoG heart rate feature in a neural network model to identify the FoG event.

5. The apparatus of claim 4, wherein the neural network model includes a FoG inertial threshold and a FoG heart rate threshold.

6. The apparatus of claim 5, wherein the FoG event is identified when the FoG inertial feature exceeds the FoG inertial threshold and when the FoG heart rate feature exceeds the FoG heart rate threshold.

7. The apparatus of claim 5, the processor further to update the neural network FoG inertial threshold based on the FoG inertial feature.

8. The apparatus of claim 5, the processor further to update the neural network FoG heart rate threshold based on the FoG heart rate feature.

9. The apparatus of claim 1, wherein the heart rate detection device includes an optical heart rate sensor to generate optical heart rate sensor data.

10. The apparatus of claim 1, wherein the heart rate detection device and the first inertial sensor are included within a device worn on a user wrist.

11. The apparatus of claim 10, further including a second inertial sensor to generate a second plurality of inertial measurements, wherein the processor identifying the FoG event is further based on the second plurality of inertial measurements.

12. The apparatus of claim 11, wherein the second inertial sensor is included within a device worn on a user ankle.

13. A Freezing of Gait (FoG) event detection method comprising:
receiving a heart rate sample from a heart rate detection device;
receiving a first plurality of inertial measurements from a first inertial sensor; and
identifying a FoG event based on the heart rate sample and on the first plurality of inertial measurements.

14. The method of claim 13, wherein identifying the FoG event includes:
extracting a FoG inertial feature; and
identifying the FoG event based on the FoG inertial feature.

15. The method of claim 14, wherein identifying the FoG event includes:
extracting a FoG heart rate feature; and
identifying the FoG event based on the FoG heart rate feature.

16. The method of claim 15, wherein identifying the FoG event includes analyzing the extracted FoG inertial feature and extracted FoG heart rate feature in a neural network model to identify the FoG event.

17. At least one machine-readable storage medium, comprising a plurality of instructions that, responsive to being executed with processor circuitry of a computer-controlled device, cause the computer-controlled device to:
receive a heart rate sample from a heart rate detection device;
receive a first plurality of inertial measurements from a first inertial sensor; and
identify a FoG event based on the heart rate sample and on the first plurality of inertial measurements.

18. The machine-readable medium of claim 17, the plurality of instructions further causing the computer-controlled device to:
extract a FoG inertial feature; and
identify the FoG event based on the FoG inertial feature.

19. The machine-readable medium of claim 18, the plurality of instructions further causing the computer-controlled device to:
extract a FoG heart rate feature; and
identify the FoG event based on the FoG heart rate feature.

20. The machine-readable medium of claim 19, the plurality of instructions further causing the computer-controlled device to analyze the extracted FoG inertial feature and extracted FoG heart rate feature in a neural network model to identify the FoG event.

21. The machine-readable medium of claim 20, wherein the neural network model includes a FoG inertial threshold and a FoG heart rate threshold.

22. The machine-readable medium of claim 21, wherein the FoG event is identified when the FoG inertial feature exceeds the FoG inertial threshold and when the FoG heart rate feature exceeds the FoG heart rate threshold.

23. The machine-readable medium of claim 21, the plurality of instructions further causing the computer-controlled device to update the neural network FoG inertial threshold based on the FoG inertial feature.

24. The machine-readable medium of claim 21, the plurality of instructions further causing the computer-controlled device to update the neural network FoG heart rate threshold based on the FoG heart rate feature.

25. The machine-readable medium of claim 19, the plurality of instructions further causing the computer-controlled device to determine a heart rate increase based on the heart rate sample and based on a plurality of heart rate historical data.
